SEQUENCE LISTING (5 sequences)

SEQ ID NO: 1 - DNA, 66 bp, Artificial Sequence
  Oligonucleotide primer
gatcctatggggcaggtgggacggcagctcgccatcatcggggacgacatcaaccgacgctatcgg

SEQ ID NO: 2 - DNA, 65 bp, Artificial Sequence
  Oligonucleotide primer
gatcccgatagcgtcggttgatgtcgtccccgatgatggcgagctgccgtcccacctgccccatg

SEQ ID NO: 3 - DNA, 20 bp, Artificial Sequence
  Oligonucleotide primer
cccccaccacttcccctctc

SEQ ID NO: 4 - DNA, 20 bp, Artificial Sequence
  Oligonucleotide primer
gcccaagctggcatccgtca

SEQ ID NO: 5 - DNA, 20 bp, Artificial Sequence
  Oligonucleotide primer
tcccgcctgtgacatgcatt

(19) United States
(12) Patent Application Publication
(10) Pub. No.: US 2006/0189558 A1
(43) Pub. Date: Aug. 24, 2006

(54) Delivery of substances to cells

(75) Inventors: Peter Francis O'Hare, Surrey (GB); Neil Douglas Brewis, Cambridge (GB); Nadia Michelle Normand, Boulogne-Billancourt (FR); Kavitha Renga Sunassee, Wimbledon (GB)

Correspondence Address: KLARQUIST SPARKMAN, LLP, 121 SW SALMON STREET, SUITE 1600, PORTLAND, OR, 97204, US

(73) Assignee: Phogen Limited

(21) Appl. No.: 11/223,449

(22) Filed: Sep. 9, 2005

Related U.S. Application Data
(63) Continuation of application No. 09/949,093, filed on Sep. 7, 2001, now abandoned.

(30) Foreign Application Priority Data
Sep. 8, 2000 (GB) 0022101.0

(51) Int. Cl. A61K 48/00; A61K 31/409; A61K 31/555

(52) U.S. Cl. 514/44; 514/185; 514/410

(57) ABSTRACT

Aggregates comprising VP22 protein and oligonucleotides or polynucleotides can be used together with a disaggregating agent (simultaneously or sequentially) to treat target cells by delivery of molecules to the cells and/or to prevent cell proliferation and/or to kill cells.

### PRIORITY CLAIM

This application claims priority from UK Application No. GB 0022101.0, filed Sep. 8, 2001, which is incorporated by reference herein in its entirety.

### FIELD OF THE INVENTION

This invention relates to aggregated compositions for delivery of substances such as nucleic acids and proteins into cells. The invention relates to the compositions and to their manufacture and use, including medical use.

### BACKGROUND OF THE INVENTION AND PRIOR ART

WO 97/05265 (Marie Curie Cancer Care: P O'Hare et al.) relates to transport proteins, in particular VP22 and homologues thereof, and to methods of delivering these proteins and any associated molecules to a target population of cells. This transport protein has applications in gene therapy and methods of targeting agents to cells where targeting at high efficiency is required.

WO 98/32866 (Marie Curie Cancer Care: P O'Hare et al.) discloses further substances and compositions related to VP22.

Elliott and O'Hare (1997) Cell, vol. 88 pp.223-233, also relates to properties and functions of VP22 protein.

The use of aluminium phthalocyanine as a sensitiser for photodynamic therapy of cancer is known (e.g. N Brasseur et al., Br J Cancer, July 1999, 80 (10), pp 1533-41).

The use of illumination of photosensitised treated cells to increase the transfection efficiency of DNA-poly-L-lysine complexes is also known (A Hogset et al., Human Gene Therapy, April 2000, 11, pp 869-880).

### SUMMARY AND DESCRIPTION OF THE INVENTION

The present invention provides uses of aggregates comprising VP22 protein, or another polypeptide with the transport function of VP22, and oligonucleotides or polynucleotides. Such aggregates can have a therapeutic function, as described below. The aggregates can be used in combination with disaggregating agents, as described below. The aggregates can be used together with disaggregating agents in the manufacture of compositions for the treatment of disease and/or for the treatment of cells to prevent their proliferation, or to kill cells. The treatment can involve delivery of the compositions to cells and subsequent disaggregation of the aggregates within the cells.

Also provided by the invention is a combination or composition comprising (a) such aggregates and (b) a disaggregating agent as described below, for use in the delivery of proteins or polynucleotides to cells.

The invention thus provides a product comprising (a) an aggregate composition which comprises VP22 (or a polypeptide with the transport function of VP22) and oligonucleotides or polynucleotides and (b) a disaggregating agent which can promote disaggregation of the aggregates in cells, as a combined preparation for administration of the components (a) and (b) either sequentially or together, for use in therapy to treat disease, and/or to treat cells by delivery of molecules to cells, and/or to prevent the cells proliferating, and/or to kill them.

Also provided are pharmaceutical compositions comprising products as described above in combination with a pharmaceutically acceptable excipient.

The invention also provides a method of delivering substances, e.g. polypeptides, peptides or polynucleotides, or antibodies, e.g. therapeutic antibodies to target cells in vitro, comprising delivering to target cells, e.g. for use in therapy to treat disease, and/or to treat cells by delivery of molecules to cells, and/or to prevent the cells proliferating, and/or to kill them, (a) an aggregated composition comprising VP22 protein (or a fragment thereof with the transport function of complete VP22 protein, or another polypeptide with the transport function of VP22) and an oligonucleotide or polynucleotide, and also (b) a disaggregating agent which can promote disaggregation of the particles of aggregates in target cells. Steps (a) and (b) can be carried out simultaneously or they can be carried out sequentially. The disaggregating agent can be a photoactivator. In that case the method can include illuminating the target cells with light of suitable wavelength to activate the disaggregating agent (actinic light). Noted below are also further disaggregating agents that are not light-activated.

The invention further provides preparations of cells treated according to methods of the invention as described above and also pharmaceuticals comprising such cell preparations in combination with a pharmaceutically acceptable excipient.

Amongst the treatments provided by the invention are treatments to inhibit cell proliferation and also treatments to kill cells. Such treatments can be applied to hyperproliferative conditions, e.g. cancer, restenosis, psoriasis and scarring (e.g. scarring associated with wound healing). Other treatments provided by the invention are treatments comprising delivering therapeutic proteins or polynucleotides to cells. Such treatments can be applied to conditions associated with the absence of a protein or peptide or polynucleotide normally present in a cell, or to conditions associated with lower levels than normal of a protein or peptide or polynucleotide in a cell (compared with a corresponding normal cell of that kind).

The methods and compositions can include photoactivating agents (and their use) to promote disaggregation of the aggregates after they have entered the target cells. Suitable photoactivating agents can be chromophores that activate disaggregation on illumination with fluorescent or visible light, preferably long-wavelength light; for example a phthalocyanine-containing chromophore, for example aluminium or zinc phthalocyanine.

In certain embodiments the photoactivating agent can be other than fluorescein and its coupled derivatives, e.g. other than fluorescein isothiocyanate, and other than rhodamine and its coupled derivatives, e.g. tetramethylrhodamine isothiocyanate (TRITC). The photoactivating agents can act by producing free radicals and/or singlet oxygen. The production of singlet oxygen is believed to occur in the use of the mentioned phthalocyanines. Other agents which can produce singlet oxygen include photosensitive dyes, e.g. rose bengal and methylene blue.

When the disaggregating agent is one that can be activated by light, and when this is to be administered in vivo, it can be preferable to use an agent which is activated by light at the red end of the spectrum, e.g. by light of wavelength of about 600 nm or greater, e.g. light of wavelength of about 675 nm, since light of this wavelength penetrates tissue more efficiently than light of a shorter wavelength, e.g. light of wavelength of less than about 600 nm.

Aluminium phthalocyanine (AT) is an example of a useful disaggregating agent which is activated by light of such wavelengths. AT is known to be preferentially absorbed by tumour tissue, it is water-soluble, relatively non-toxic and is activated by light with a wavelength of about 675 nm. AT can be used as a disaggregating agent in methods and compositions according to the invention either in its unsubstituted form, or alternatively as a substituted derivative, e.g. as a sulphonated derivative, e.g. as a disulphonated derivative, or as a tetrasulphonated derivative.

Alternatively, disaggregating agents can be used which do not require light for their action (non-photoactivating disaggregating agents). Preferred examples of such agents include agents which can promote an increase in pH within cellular compartments such as endosomes. Examples of this type include tamoxifen and chloroquine. Also disaggregating agents can be used which produce pores in cellular membranes such as endosome membranes. Examples of this type include perforin and streptolysin-0. It can also be desirable to facilitate disaggregation of the components of the aggregate e.g. in the absence of any external disaggregating agent, by incorporating an agent which can promote disaggregation in certain cellular environments as a component of the aggregate, e.g. by linkage of such an agent with VP22 and/or the oligonucleotide. For example, a peptide sequence which is lytic at endosomal pH can be incorporated into the aggregate and can faciliate disaggregation of aggregates in cell endosomes, e.g. a JTS peptide incorporating a cleavable (lytic) linker sequence can be usefully used (Gene Therapy 1996, 3, pp 448-457, S Gottschalk et al.).

Disaggregating agents such as those mentioned above which can promote disaggregation of the aggregates in target cells in the absence of light can be particularly useful when it is difficult to administer light to the target cells, e.g. when the target cells are in vivo, e.g. when the target cells are in vivo and deep within the body tissues, e.g. when the target cells are deep tumours or tumour metastases.

Use of disaggregating agents which are preferentially absorbed by tumour tissue, e.g. phthalocyanine containing compounds, e.g. aluminium phthalocyanine or zinc phthalocyanine, can be preferred when the cells to be treated are cancer cells or other hyperproliferating cells.

The disaggregating agents can form part of a composition with the aggregates or they can be administered separately from the aggregates. Suitable methods of administration are described below.

Compositions according to the invention can be in pharmaceutically acceptable form suitable for delivery to cells whether ex-vivo, or in culture, or in-vivo, e.g. as a sterile composition comprising pharmaceutically acceptable excipients.

The aggregates can be made by mixing oligonucleotides or polynucleotides with VP22 protein or equivalent. The resulting particle sizes can be in the range 0.1-5 microns e.g. 1-3 microns.

Ratios of between 2:1 and 1:1 of protein to nucleotide are most preferred for formation of aggregates. Higher ratios of protein can be used, but lower ratios are less preferred.

By aggregates we mean associations of molecules forming particles for example particles of 0.1-5 microns in size e.g. of 1-3 micron in size. ‘Aggregate’ here is not intended to imply a state of denaturation or inactivity: the aggregates usefully contain active protein and/or functionally active oligo- or polynucleotides.

Oligo- or polynucleotides suitable for forming part of the aggregates of the invention can preferably comprise at least 10 bases(nucleotides) and in length can range widely in size (e.g. in the range 10-50 e.g. 20) e.g. they can be about 4 kilobases in size, and they can comprise plasmids, mini-circles of DNA, or single or double stranded DNA or RNA, or other functionally active nucleotide sequences. Optionally, the nucleotide sequences can also be associated with a DNA condenser, e.g. protamine sulphate.

The VP22 protein referred to can be the native VP22 protein of HSV1 or HSV2, or it can be a homologue as mentioned in WO 97/05265 (Marie Curie Cancer Care: P O'Hare et al.). For example, it can be a VP22 homologue from bovine herpesvirus. Alternatively, the aggregates can comprise a protein with a sub-sequence less than the whole sequence of the wild-type VP22 protein, that retains the transport functionality of wild-type VP22 protein. Such a sub-sequence can be, for example, a protein corresponding in sequence to amino acid residues 159-301 of VP22. Native VP22 is believed to form stable multimers readily, either dimers or tetramers. The sub-sequence based on amino acids 159-301 of VP22 is believed to form dimers readily. The VP22 protein, or protein based on a functional sub-sequence, can further comprise other sequences, e.g. at least one flanking tag fused at the N terminus or at the C terminus of the VP22 or sub-sequence. The tag can be for example, a T7 tag which is an example of an epitope tag enabling antibody detection, e.g. at the N terminus, or it can be for example, a his tag which enables purification of the protein on a nickel containing column, e.g. at the C terminus.

The oligonucleotides or polynucleotides contained in the aggregated composition can be DNA or RNA, that is the nucleotides contained therein can have either an RNA structure wherein the sugar is ribose, or they can have the structure found in DNA wherein the sugar is deoxyribose. For example, oligonucleotides or polynucleotide component can be a circular plasmid. When the nucleotides forming the aggregates are RNA, the ribose sugar can be 2′-O-methylated for increased nucleotide stability. In certain examples, the nucleotides can comprise negatively charged modified derivatives of nucleotides e.g. phosphonate derivatives or phosphorothioate derivatives.

Additional molecules for delivery to a cell can be linked to the protein and/or nucleotide components of the aggregates by means of a linking agent. For example, molecules for delivery to cells, e.g. peptides, drug molecules, or therapeutic antibodies can be coupled to the oligonucleotide, e.g. by an ester linkage. Or they can be chemically cross linked to VP22 using standard known techniques, e.g. molecules for delivery can usefully be cross linked onto exposed cysteine or amine residues of VP22. Alternatively, VP22 can be expressed as a fusion with an Intein protein using standard techniques, the pH of the VP22-Intein fusion protein can then be decreased e.g. to about pH 7.0, or the protein can be exposed to a reducing agent, the Intein will cleave off the VP22 leaving an N-terminal cysteine residue which can be used to chemically couple molecules to VP22 (J. Biol. Chem, 1999, 274 (26), pp 18359-18363; Intein expressing plasmids are available from New England Biolabs, Beverly, Mass., USA). Optionally, the linking agent can be biotin and the aggregates can form part of a streptavidin-biotin complex in which the oligo- or polynucleotide is labelled with biotin, e.g. at the 5′ end, and this can then be mixed with streptavidin, e.g. streptavidin Alexa 594 (TM), which is streptavidin bound to a fluorophore molecule. Preferably, the streptavidin molecule is modified so that it can be coupled to a molecule, e.g. a drug, which it is desired to deliver to cells, e.g. so that it comprises a disulphide bond which can be used to link it to a molecule which it is desired to deliver to cells and thereby promote subsequent release of the molecule within the cell by intracellular cleavage of the disulphide bond.

Aggregates containing nucleotides such as phosphorothioate derivatives can be of good stability in serum, in spite of the presence of Dnases in serum. They can also be stable in high concentrations of denaturants such as urea, e.g. 7M urea.

Where the oligo- or polynucleotides contain phosphorothioate or other modified nucleotide units as mentioned above, they can be especially stable against degradation by components of serum.

The oligo-or polynucleotides contained in the aggregated compositions can contain ordinary nucleotide phosphodiester linkages. Alternatively, e.g. for achieving longer life and stability against hydrolysis, they can contain phosphorothioate linkages in place of phosphodiester linkages, or they can contain a mixture of phosphorothioate and phosphodiester linkages with the phosphorothioate linkages present at the end of the molecule.

It can also be useful to label the oligo- or polynucleotide, for example with a detectable label to facilitate detection and monitoring of the aggregate. The label can be at either the 5′ or at the 3′ end of the synthetic nucleotide. For detection or monitoring of the aggregate any label capable of detection can be used, such as radio-label, or a fluorochrome label.

The nucleotide can be a fluorescent-labelled 20 base oligonucleotide (20-mer) containing phosphorothioate linkages. It can be labelled at the 5′ end with 5′ fluorescein phosphoroamidite (Genosys), or at the 3′ end with fluorescein (Genosys), or at the 5′ end with a terminal fluoresceinyl-base (Life Technologies). Also usable is a Texas Red labelled 20 mer phosphorothioate that is labelled at the 5′ end or 3′ end with Texas Red (Genosys).

Aggregates according to the invention can be used to deliver their constituents into target cells.

Cells to which the aggregates and disaggregating agents and optionally actinic light can be delivered can be cells of a tissue or an organ in a mammalian subject e.g. a human subject, or they can be explanted cells, or they can be cultured cells e.g.for production of a desired protein. Cultured cells that can be used include but are not limited to: CHO, COS, HeLa and Vero cells, primary cells such as rat aortic smooth muscle cells (RASMC; obtainable from the American tissue culture collection (ATCC)) and human primary cells e.g. aortic smooth muscle cells (HASMC; obtainable from the ATCC), and human neuronal and epithelial primary cells, T24 human bladder carcinoma cells (obtainable from the ATCC), RAW 246 macrophage cells, A549 human caucasian lung carcinoma cells (obtainable from the European collection of cell culture), KB-3-1 human cervix carcinoma cells (derived from HeLa cells and obtainable from German collection of cell cultures (DSMZ)), and KB-vl human cervix carcinoma cells (derived from HeLa cells and obtainable from German collection of cell cultures (DSMZ)).

In certain examples, when the aggregate comprises a protein or peptide fused to VP22, or to a sub-sequence thereof, the protein or peptide can be any which can generate an antibody or CTL immune response. Thus the aggregates can be immunogenic compositions, for example they can be vaccines, e.g. DNA or protein vaccines, or both. It can be particularly useful to improve vaccine potency for the VP22 to be linked to the antigen which it is desired to deliver to a subject, e.g. as part of a VP22 fusion protein.

In certain examples, the VP22 protein can usefully be a fusion protein in which the protein fusion partner possesses enzymatic activity. For example, a VP22-thymidine kinase (TK) fusion protein, can be used in the aggregated compositions e.g. where the target cells are cancer cells e.g. neuroblastoma cells. The aggregated compositions and disaggregating agents and optionally actinic light can be delivered to target cells, and this can be followed by treatment of the target cells with ganciclovir or equivalent drugs, whereby the TK activity in the composition transported into the cell activates the ganciclovir for cell killing in per se known manner.

It can also be useful to deliver proteins of the aggregates for corrective protein therapy.

It can also be useful where VP22, or a sub-sequence thereof, is fused to a cell targeting molecule, e.g. a peptide that binds to a cell surface receptor, to facilitate cell specific targeting of the complex. For example, VP22 can be fused to a tumour targeting molecule such as transferrin, or folate. Alternatively, VP22, or a sub-sequence thereof can usefully be fused to a peptide comprising an amino acid sequence which consists of the amino acids arginine, followed by glycine and aspartate (also known as an RGD motif; SL Hart, et al., 1996, Gene Therapy 3, pp 1032-1033) and used to target epithelial and endothelial cells. Alternatively, VP22 can be conjugated, using standard methods known in the art for conjugation of sugars to proteins some of which are described in N Sdiqui et al., 1995, Drug delivery 2, pp 63-72 and E Bonifils et al., 1992, Bioconjugate Chemistry 3, pp 277-284, e.g. to a glycoside or lectin molecule such as those mentioned in N Sdiqui et al., 1995, Drug delivery 2, pp 63-72 and E Bonifils et al., 1992, Bioconjugate Chemistry 3, pp 277-284, to facilitate targetting of certain lectin expressing cells, e.g. lectin expressing tumour cells, macrophages, hepatocytes and parenchymal cells.

It can also be particularly useful for the disaggregating agent to be linked (e.g. covalently) to a cell targeting substance, for example a monoclonal antibody which can bind to the chosen target cells, e.g. cancer cells of a desired target type. For example, sulphonated aluminium phthalocyanine can be linked to a monoclonal antibody, e.g. one which binds to the overexpressed tumour marker carcinoembryonic antigen (CEA) as described by M Carcenac et al., in Photochem. Photobiol., 1999, 70 (6): pp930B6).

The oligonucleotide or polynucleotide contained in the aggregated composition according to the invention can be a substance which it is desired to deliver to a target cell.

For example, the oligonucleotide or polynucleotide can be single stranded DNA or RNA, such as a 20 mer, and it can have a base sequence that enables it, or its transcription product, to function as an antisense or ribozyme molecule in per se known manner, in effect to suppress functional expression of a chosen gene. For example the polynucleotide can be the synthetic hammerhead ribozyme, or any functional homologues or modifications thereof, which can recognise and cleave c-myb RNA, and thereby inhibit cell proliferation (Jarvis et al., J. Biol. Chem., 1996, 271, 29107-29112).

Alternatively, the oligo- or polynucleotide can be antisense in sequence, e.g. it can be antisense to the c-myb gene which is associated with cell proliferation, or e.g. antisense to the p27 gene to prevent smooth muscle cell proliferation, or e.g. antisense to a protein which inhibits apoptosis, such as the Bcl protein, or antiviral antisense e.g. antisense which can bind to a viral AUG start codon or anti-HIV antisense which is complementary to a region of the HIV gag mRNA (J Lisziewicz et al., 1994, PNAS 91, PP 7942-7946), or antitumoral antisense, e.g. antisense to the ras oncogene (G. Chen et al., 1996, J Biol. Chem. 271, pp 28259-28265), or it can be antiparasitic antisense, e.g. trypanasome antisense (P. Verspieren et al., 1987, Gene 61, pp307-315). Alternatively, the oligo- or polynucleotide can have the function of correcting splicing defects. The oligo- or polynucleotides can also usefully be chimeroplasts, which are chimeric RNA/DNA oligo- or polynucleotides and which can correct mutations. The oligo- or polynucleotides can also usefully be DNA encoding endogenous ribozymes. The oligo- or polynucleotides can also be RNA which can function as an interfering RNA and prevent transcription of a target gene. It can also be useful to deliver decoy oligonucleotides to cells, such oligonucleotides can prevent proteins binding to their binding sites, for example it can be useful to deliver a decoy oligonucleotide which binds to E2F and prevents E2F binding to its receptor, and this can inhibit vascular smooth muscle cell proliferation in vivo (Proc. Nat Acad. Scien., 1995, 92, pp 5855-5859, R Morishita et al.). The oligonucleotide for delivery to cells can also usefully be CpG- containing oligonucleotides, and these can function as vaccine adjuvants (Antisense and Nucleic Acid Drug Development 1998, 8, pp 181-184, DM Klinman).

In other examples, the oligonucleotide or polynucleotide can be single stranded DNA of appropriate sequence to enable it to bind to a specific sequence of DNA in the target cell, by forming a triple helix in per se known manner, to block transcription of the gene to which the nucleotide has bound.

In further examples, the oligonucleotide or polynucleotide can be double stranded DNA and can be of appropriate sequence to function as a binding site that binds a specific transcription factor in a target cell, thereby sequestering the transcription factor in the cell (in per se known manner) and suppressing expression of genes that depend for expression on the sequestered transcription factor.

Alternatively or additionally, the protein contained in the aggregated composition according to the invention can be a substance which it is desired to deliver to a target cell. For example, it can comprise VP22 or a protein comprising sub-sequence thereof, or a fusion protein comprising VP22, e.g. for use as a vaccine.

The aggregated compositions can also comprise further or other substances for delivery to target cells, such as nucleotides, proteins or peptides fused to VP22.

For example, the aggregated composition can comprise and deliver to a target cell circular or linear DNA of a size sufficient to encode a gene, e.g. to encode a protein. The delivered DNA can also comprise the necessary gene expression elements needed for its expression in the target cell.

In certain examples, the aggregated composition can comprise and deliver single stranded mRNA molecules, of size sufficient to be translated into a protein or peptide, into the cytoplasm of a target cell where the mRNA can be translated into protein or peptide.

In a further aspect of the invention, the VP22 component of the aggregate contains a VP22 sequence and a further component, which can be either the remaining part of a fusion protein, a protein sequence of a desired functionality which it is desired to deliver within the target cell or a nucleotide sequence which it is desired to deliver within the target cell.

The further component can be linked to the VP22 by a cleavage-susceptible amino acid sequence which is susceptible to cleavage by intracellular protease within the target cell. The proteolytic site can be e.g. a site cleaved by a virus encoded protease, such as for example an HIV-encoded protease (D. Serio et al., 1997, PNAS 94, pp 3346-3351) so that cleavage only occurs in virus infected cells, or alternatively the cleavage site can be one which is only cleaved by a cell-specific protease, thereby enabling delivery to a specific cell type. In this aspect of the invention, the fusion protein or coupling product can be delivered within the target cell and cleaved there by protease to release the coupling partner of the VP22, that is, the chosen protein or the nucleotide.

It can also be useful in certain examples to include a coupled protein product that is only active after cleavage of the coupled product in the target cell.

Fusogenic peptides, which can facilitate release from endocytic vesicles within the cell, can also be present in the aggregates according to the invention, e.g. influenza haemagluttinin for intracellular delivery. Peptides which can facilitate intracellular targetting can also usefully be present in the aggregates, e.g. the NES peptide (nuclear export signal; L Meunier et al. 1999, Nucleic Acids Research 27, pp 2730-2736), e.g.a peptide termed the KDEL peptide (S Seetharam et al., 1991, J Biol Chem 266, pp17376-17381 and U. Brinkmann et al., 1991, PNAS 88, PP8616-8620).

It can also be useful to modify the oligo- or polynucleotide so that it can be coupled to a molecule which it is desired to deliver to a cell, for example through a disulphide bridge which can be reduced within the cell and thereby facilitate release of the molecule for delivery.

Compositions according to the invention can be delivered to target cells in vivo, such as cells of a tissue or an organ in a mammalian subject, e.g. a human subject. It can for example, be advantageous to deliver compositions according to the invention to cancer cells, e.g. to introduce an antisense molecule which is of appropriate (per se known) sequence to target a chimeric oncogene, or to suppress a cancer gene, e.g. ras or p53, or to suppress an anti-apoptotic gene such as a member of the Bcl gene family.

The disaggregating agents can form and be administered either (a) as part of a composition with the aggregates or (b) they can be administered separately not forming part of the same composition.

Compositions according to the invention can be delivered to target cells in vivo, by for example, direct injection into the target cells, for example into a tumour cell mass, or the compositions can be delivered to target cells in vivo by systemic administration, e.g. by using a catheter.

Compositions according to the invention can also be formulated using per se known methods for topical delivery, e.g. for use as part of a therapy for psoriasis, eczema or skin cancer. Alternatively, the compositions can be encapsulated into slow release capsules e.g. suitable for oral delivery using standard methods well known in the art. For example, the compositions of the invention can be encapsulated into two-component slow release capsules, e.g. when the aggregates are within one compartment and the disaggregating agent is in the other compartment, such a capsule can break down in vivo thereby releasing the two components both together.

The compositions can also be associated with other delivery systems, for example they can be coupled to liposomes, such as cationic liposomes. It can be particularly useful if the aggregates are associated with condensing agents, such as DNA condensing agents, e.g. hydrophilic polymers. Among suitable condensing agents are protamine sulphate, and DNA condensing agents such as poly-lysine and histones.

When compositions described herein are administered to target cells within a subject, both the aggregate particles and disaggregating agent need to be present together, so that the disaggregating agent can promote disaggregation of the aggregates within the target cells of the subject. When a composition of aggregate particles and a disaggregating agent are administered separately to the subject the necessary proximity can be achieved by administering both components at the same time or immediately one after the other, e.g. by administration of each component separately at the same locus, or at a closely neighbouring locus, or alternatively by administering each component separately at different loci at the same time or immediately one after the other.

Thus for example, when the aggregates and the disaggregating agent do not form part of the same composition and are used in combination by being administered separately, both agents can be administered at the same locus or at a closely neighbouring locus, e.g. both can be administered by direct injection into the chosen target cells, e.g. into a tumour cell mass. Alternatively, both agents can be administered at different loci, for example the aggregates can be delivered by direct injection into the chosen target cells and the disaggregating agent can be administered systemically.

Alternatively, the aggregates and disaggregating agent can be administered separately to a subject at different times at the same locus, or at a closely neighbouring loci, or at very different loci (as described above). For example, the aggregates can be administered to a subject before administration of the disaggregating agent, e.g. within about 4 days before, e.g. within about 2 days, or within about 1 day, before, or in certain cases within a few hours before.

Alternatively, the disaggregating agent can be administered to a subject before administration of the aggregates, e.g. within about 120 hours before, e.g. within about 48 hours, and possibly within about 4 hours before administration of the aggregates.

When aluminium phthalocyanine is the disaggregating agent and it is administered to a subject in vivo, e.g. by systemic delivery to a human subject, up to about 100 mg/kg (body wt) can usefully be administered to the subject. However, when it is not desired to kill target cells in vivo by administration of the aggregates and aluminium phthalocyanine and actinic light, it can be desirable to administer to a subject less than about 100 mg/kg (body wt) of aluminium phthalocyanine, e.g. about 50 mg/kg (body wt) or less.

When the disaggregating agent is activated by light, activation can be achieved by illumination at the target site with actinic light for a time period from about a few seconds up to about minutes, e.g. up to about 10 minutes. When it is not desired to kill target cells it can be desirable to illuminate the target cells for a short period of time, e.g. less than about 10 minutes, e.g. less than about 5 minutes or less than about 2 minutes or about a few seconds.

When aluminium phthalocyanine is the disaggregating agent the wavelength of actinic light used is about 633 nm and the target cells are illuminated for up to about 10 minutes, when it is desired to kill the cells. When it is not desired to kill the target cells they can be illuminated for a shorter period of time, e.g. less than about 10 minutes, e.g. less than about 5 minutes or less than about 2 minutes, or as little as a few seconds.

It can be especially useful to administer tamoxifen as the disaggregating agent when the cells to be treated are cancer cells in vivo, e.g. breast cancer cells. It can be useful to administer up to about 80 mg of tamoxifen in a single dose to a subject, e.g. to a human subject.

Compositions according to the invention can be formulated according to known methods for therapeutically useful compositions, whereby the compositions are combined in admixture with a pharmaceutically acceptable carrier.

The VP22 component of the aggregates can be stored for long periods at −70 deg C., for example in a solution of PBS, or alternatively it can be lyophilised and re-constituted before use. The oligonucleotide component of the aggregates can be stored for long periods at −20 deg C. or at 4 deg C., for example in a solution of Tris buffer (pH 7.0 or preferably pH7.5). The VP22 and oligonucleotide components can then be mixed at room temperature for at least 10 mins to enable formation of aggregates according to the invention just prior to delivery of aggregates to cells.

Compositions according to the invention can be delivered to target cells which are cells cultured in vitro, for example to CHO, COS, HeLa and Vero cells. The cultured cells containing the aggregates can be used, for example, for target validation in in-vitro testing of gene expression products.

In other embodiments, cells treated with compositions according to the invention can be explanted cells and can then be re-introduced in vivo, e.g. into a mammalian subject.

The aggregates can be substantially resistant to trypsinisation of cultured cells containing them. Therefore cells containing the aggregates in culture can be trypsinised prior to use.

When the target cells are cultured cells and a disaggregating agent is used which is a photoactivating agent it can be useful to produce a cell suspension prior to illumination of the cells, e.g. by trypsinisation of the cells in culture using per se known methods, as cells in suspension can be illuminated for a shorter time period than adherent cells to promote disaggregation of the aggregates.

When the disaggregating agent is a photoactivating agent irradiation can be achieved in vivo, for example, by introducing into a patient to be treated an endoscope comprising laser optic lines for emitting radiation. Dissociation of aggregates can also be facilitated in the absence of light by introduction of a cleavage site, such as a protease site, or a fusogenic peptide, e.g. the FLU fusion peptide.

Aggregates described herein can be useful as cell delivery systems for substances such as proteins or nucleotides, fused with VP22 protein, or a functional part thereof, and can enable delivery into target cells of large amounts of protein or nucleotides.

Following exposure of a cell population to such aggregates, they can be taken up by the cells and the VP22 fusion protein can cause transport to the cell nucleus.

Once the aggregates are taken up into a cell they have been observed in certain examples to remain within the cell for some days, and can also resist cell trypsinisation.

Aggregates described herein can be made using a method comprising (a) mixing a VP22 protein or a suitable equivalent thereof as mentioned above, optionally fused or covalently coupled to a protein sequence or a nucleotide for delivery to a target cell, with an oligonucleotide or polynucleotide, followed by (b) incubating the mix obtained in step (a).

The invention also provides a method for transporting substances into cells, comprising contacting target cells with an aggregated composition.

Examples of the invention are described below without intent to limit its scope.

### EXAMPLE 1

This example describes killing target cells by delivering to the target cells (i) an aggregated composition which comprises a fragment of VP22 consisting of amino acids >159-301′ present as a fusion protein with the BH3 domain of the bak protein, and also an FITC-labelled ICAM oligonucleotide, and also (ii) a disaggregating agent which is the photoactivator aluminium phthalocyanine, followed by activation of the aluminium pthalocyanate with actinic light.

The BH3 domain of the bak protein can induce cell apoptosis and is a functional homologue of the BH3 domain of the bax protein (EP Hollinger et al., 1999, J. Biol. Chem., 274, (19), PP 13298-13304).

The 159-301-BH3 fusion protein can be prepared for example as follows:

159-301 protein can be made in an E.coli expression system expressing a plasmid encoding 159-301 protein, which is a PET-based plasmid containing an IPTG sensitive promoter. The his tag is placed at the C terminus of the protein.

A double stranded oligonucleotide with the following sequence corresponding to BH3 can be made and cloned into the Bam HI site of the VP22>159-301′ expression plasmid used to encode the VP22>159-301′ protein, as mentioned above.

5′GATCCTATGGGGCAGGTGGGACGGCAGCTCGCCATCATCGGGGACGACATCAAC
CGACGCTATCGG
5′GATCCCGATAGCGTCGGTTGATGTCGTCCCCGATGATGGCGAGCTGCCGTCCCAC
CTGCCCCATG

The above strands are complementary such that the sequence of the first strand from the seventh residue (adenine) in the 5′ to 3′ direction is complementary with the sequence of the second strand from the second residue from the end (thymine) in the 3′ to 5′ direction.

50 ml of bacterial culture expressing the plasmid mentioned above can be grown in nutrient broth suitable for the growth of E. coli, such as L nutrient broth (Oxoid), and also containing kanamycin and chloramphenicol. The recombinant bacteria can be induced by addition of IPTG (0.5 mM) to a logarithmic phase culture, and the cells harvested by centrifugation (6000 rpm, 4 degC., 20 min). After pelleting the cells can be resuspended in 40 ml of cold lysis buffer containing: 50 mM sodium phosphate (pH 8.0), 300 mM sodium chloride, 5 mM imidazole (pH 8.0), 5 mM beta-mercaptoethanol, 1 microg/ml of leupeptin, lmicrog/ml pepstatin and 1 mg/ml lysozyme.

The lysis mixture can be incubated for 30 min with occasional shaking, and is then sonicated on ice three times for 15 seconds followed by addition of 0.1 % NP-40. Dnase and Rnase can then be added to 10 microg/ml and incubated on ice for 20 min with occassional shaking. The lysate can then be drawn through a narrow gauge syringe three times. This can be followed by centrifugation of the lysate at 20,000 rpm for 15 min at 4 degC. The supernatant containing the VP22-BH3 fusion protein can be retained. The BH3-VP22>159-301′ fusion protein can be purified as follows:

The protein can be enriched on DEAE sepharose (Pharmacia) by using a batch method, in the presence of lysis buffer comprising 50 mM sodium phosphate (pH 8.0), 300 mM sodium chloride, 5 mM imidazole (pH 8.0), 5 mM beta-mercaptoethanol, 0.1% NP-40, and 1 microgram/ml leupeptin and 1 microgram/ml pepstatin.

The supernatant can then be further purified on nickel-NTA beads in a batch method. Protein can be bound to the beads at 4 degC. for 1 h. The beads can then be washed three times for 30 mins in wash buffer of the same composition as lysis buffer except that it contains 10% glycerol, 0.1% NP-40, 40 mM imidazole (pH 8.0). Bound protein can then be eluted three times in 1 ml of eluate buffer each time. The eluate buffer can have the same composition as lysis buffer except that it contains 10% glycerol, 0.1% NP-40, 500 mM imidazole (pH 8.0). The eluate buffer can then be exchanged by PD-10 sephadex column chromatography into PBS, 10% glycerol, 5 mM B-mercaptoethanol.

The BH3-VP22>159-301′ fusion protein obtained by the method described above can be used in the formation of aggregated compositions. Alternatively, it can be dialysed for 12 hours in PBS before use.

Aggregates can be produced as follows:

25 microlitres of 20 mer phosphorothioate-linked oligonucleotide (lOmicromolar solution in PBS) is added to 25 microlitres of 159-301-BH3 protein solution in PBS (20 micromolar solution containing approximately 150 mM sodium chloride and 10 mM phosphate and at a pH between 7 and 7.2). The oligonucleotide is labelled at the 5′ end with fluorescein and has a base sequence as follows:
5′ CCC CCA CCA CTT CCC CTC TC 3′.

This sequence is commercially available and is complementary to a segment of mRNA encoding an intracellular-adhesion molecule, or ICAM. In the aggregates produced the final concentrations of protein and oligonucleotide in 50 microlitres of solution are about 10 micromolar protein and 5 micromolar oligonucleotide.

The mixture is mixed and left for at least 10 min at room temperature. Fifty microlitres of this mixture is then added to 450 microlitres of tissue culture medium (with or without added) serum and can be stored at about 4 degC. Aggregates are pre-warmed before addition to cells.

The formation of the aggregates of the invention can be monitored by using microscopy e.g. phase contrast or fluorescence microscopy, or by agarose gel electrophoresis of the aggregates.

Aggregates can be delivered to cells as follows:

Fifty microlitres of aluminium phthalocyanine (0.2 mg/ml solution in Dulbecco's modified Eagles medium (DMEM), obtained from Sigma, and containing 10% foetal calf serum (FCS)) can be added to the 500 microlitres of solution containing the aggregates (produced by the method previously described) and this solution can then be added to cultured COS cells (about 4×10ˆ4 cells in A4 chamber slides@).

The COS cells can then be incubated for about 20 hours at a temperature of 37 deg C. At the end of incubation the COS cells can be illuminated with light from a 633 nm laser under a confocal microscope for about 2 minutes. The FITC fluorophore present in the aggregates does not absorb light at 633 nm, only the aluminium phthalocyanine is activated by light at 633 nm. The COS cells and aggregates can then be observed under a confocal microscope with light from an attenuated 488 nm laser. The FITC fluorophore does absorb light at 488 nm, thereby enabling monitoring of the aggregates, but attenuation of the laser ensures that the FITC cannot be activated by this light. Re-distribution and disaggregation of the aggregates can be observed within the cells, and is followed by cell death.

### EXAMPLE 2

This example describes killing target cells by delivering to said cells a medicament as described in example 1, except that the disaggregating agent is tamoxifen which is a non-photoactivating agent and thus light is not used to activate the tamoxifen. Aggregates can be made and delivered to COS cells as described in example 1 except that the 500 microlitres of solution containing the aggregates (produced by the method previously described in example 1) is added to the cells without addition of tamoxifen. The COS cells can then be incubated overnight at 37 deg C. The following day a solution of tamoxifen (10 micromolar final concentration in DMEM containing 10% FCS) can be added to the cells and the cells incubated overnight at 37 deg C. The COS cells and vectosomes can then be observed under a confocal microscope using light from a 488 nm attenuated laser (as described in example 1). Re-distribution and disaggregation of the aggregates within the cells can be observed, and is followed by cell death.

### EXAMPLE 3

This example is similar to Example 2, except that the disaggregating agent is chloroquine (100 micromolar final concentration in DMEM containing 10% FCS). Re-distribution and disaggregation of the aggregates within the cells can be observed, and is followed by cell death.

### EXAMPLE 4

This example describes delivery of aggregates to cells in vivo. Aggregates are prepared as described in example 1, except that the fragment of VP22 consisting of amino acids >159-301′ is used instead of the VP22BBH3 fusion, the fluoroscein labelled oligonucleotide is mixed ICAM oligonucleotide radiolabelled at the 5′ end with S 35, and both the VP22 and oligonucleotide are mixed together at four times the concentrations of those used in example 1.

Aggregates prepared as described above are then injected (by a single intravenous dose of 0.05 ml volume of aggregate solution) into the tail veins of male CD1 mice (obtained from Charles River Limited, UK). Following administration of the aggregates animals were observed for signs of distress. Twenty four hours after administration of the aggregates to the mice, the mice are sacrificed. At sacrifice, brain, heart, liver kidney, lung, small and large intestines, spleen and stomach are removed and snap frozen in liquid nitrogen. Total radioactivity of the tissues is then measured by solubilising and decolourising the tissues and counting in a scintialltion counter after addition of the scintillation cocktail according to standard methods in the art. All tissues tested were found to be radioactive, and higher levels of radioactivity were found in tissues of mice injected with aggregates than in control mice injected with radioactive oligonucleotide alone.

This demonstrates that aggregates can persist in tissues in vivo for up to 24 hours following intravenous administration. Also, aggregates do not appear to be toxic to mice since none of the mice were distressed or died in the 24 hours prior to sacrifice.

### EXAMPLE 5

This example describes delivery of aggregates to CT 26 tumour cells in vivo by direct intra-tumoural injection.

Test aggregates are prepared as described in example 1, except that the fluorescent label on the ICAM oligonucleotide is BODIPY 630/650 (obtainable from IBA GmbH, Gottingen, Germany), and both the VP22 >159-301′-BH3 fusion protein and oligonucleotide components are mixed together at four times the concentrations of those used in example 1.

Fifty microlitres of the aggregate solution is then directly injected into CT26 tumours in mice. Following administration of the aggregates the mice are observed for signs of distress. Twenty four hours after administration of the aggregates to the mice, the mice are anaesthetised and half of the CT 26 tumours are illuminated for 10 minutes using a cold light source KL2500 LCD (from Schott, Wiesbaden, Germany) on the maximum setting, using OG550 and Sp700 filters (from Melles Griot, Irvine, USA) and illuminating an area of about 2.1 cm diameter about 1 cm above the tumour. Twenty four hours after illumination of the tumours, the tumours are removed from the mice and are frozen in an isopentane dry ice bath. Controls included mice injected with PBS, or a BH3 peptide, or aggregates formed using VP22>159-301′ protein and ICAM oligonucleotide instead of the aggregates described above.

Presence of aggregates in the CT 26 tumours can be observed using fluorescence microscopy which detects the fluorescent oligonucleotide component of the aggregates. Test aggregates were also found to induce significant apoptosis of the CT26 tumour cells in comparison to controls following excision of the tumours from the mice. Apoptosis was detected using the DermaTACS (TM) in situ apoptosis detection kit available from R&D systems (Minneapolis, USA).

### EXAMPLE 6

This example describes delivery of aggregates in vivo by injection of CT 26 tumour cells pre-loaded with aggregates.

Test aggregates are prepared as described in example 5, except that the ICAM oligonucleotide is replaced by the Bodipy labelled ISIS 2302 oligonucleotide.

The oligonucleotide is labelled at the 5′ end with BODIPY 630/650 and has a base sequence as follows:
5′GCCCAAGCTGGCATCCGTCA.

This sequence is commercially available from IBA GmbH, Goettingen, Germany.

Two hundred and fifty microlitres of this aggregate solution is then added to about 4×10ˆ5 CT26 cultured cells in vitro. The cells are then cultured in vitro for a further 24 hours, this is followed by changing the DMEM cell growth medium. About 0.2 ml of these CT 26 cells (approximately 5×10/4 cells) is then injected into the flank of anaesthetised mice and the point of injection clearly marked. Twenty fours hours later mice are anaesthetised and the area of skin around the point of injection is illuminated for 10 mins using a cold light source KL2500 LCD (from Schott, Wiesbaden, Germany) on the maximum setting, using OG550 and Sp700 filters (from Melles Griot, Irvine, USA) and illuminating an area of about 2.8 cm diameter about 1 cm above the tumour). Eight days after illumination tumour growth is measured. Controls included mice injected with PBS alone, or with aggregates which do not encode the BH3 peptide.

It was observed that the test aggregates are activated by light in vivo and they produce a significant reduction in the size of the tumour in comparison to controls.

### EXAMPLE 7

This example describes delivery of aggregates encoding anti-raf antisense oligonucleotide to A549 tumour cells in vivo by direct intra-tumoural injection.

Test aggregates can be made according to example 1, except that the fragment of VP22 consisting of amino acids >159-301′ is used instead of the VP22BBH3 fusion, and the oligonucleotide is an anti-raf oligonucleotide.

The oligonucleotide is labelled at the 5′ end with BODIPY 630/650, and it has a base sequence as follows:
5′ TCCCGCCTGTGACATGCATT 3′

This sequence is commercially available from IBA GmbH, Goettingen, Germany.

Fifty microlitres of this aggregate solution is then directly injected into A549 subcutaneous tumour cells in mice. Two injections of aggregates are given every week for a total of 4 weeks. Twenty four hours after each injection the mice are anaesthetised and the A 549 tumour illuminated for 10 minutes using a cold light source (KL1500, using an OG550 and SP700 filter, and illuminating a tumour area of about 2.8 cm diameter about 1.6 cm above the tumour). Controls were mice injected with PBS alone, an inactive oligonucleotide, oligonucleotide alone or with the aggregates as described above but in the absence of illumination of the tumour.

A significant decrease in tumour growth was observed only in the presence of the test aggregates and illumination. No decrease in tumour growth was observed in the control mice.

### EXAMPLE 8

This example describes delivery of aggregates encoding anti-raf antisense oligonucleotide to A549 tumour cells in vivo by direct intra-tumoural injection.

Aggregates are made as described in example 7, except that the oligonucleotide is labelled at the 5′ end with fluorescein.

One hundred microlitres of the aggregate solution is then directly injected into A549 subcutaneous tumour cells in mice. At various time points after injection into the mice (at 15 minutes, 2 hours, 8 hours, 24 hours and 4 days after) tumours are excised and frozen. Tumours are then prepared for cryostat sectioning using standard methods known in the art. The sections are examined by fluorescence microscopy to detect the aggregates.

Aggregates were observed in all of the tumours excised, thus demonstrating persistence for up to 4 days.

The present disclosure extends to modifications and variations of the description given herein that will be apparent to the reader skilled in the art. The disclosure hereof, incorporating WO 97/05265 (P O'Hare et al.), WO 98/32866 (Marie Curie Cancer Care: P O'Hare et al.) and Elliott and O'Hare (1997; cited above) which are made an integral part hereof, is intended to extend in particular to classes and subclasses of the products and generally to combinations and sub-combinations of the features mentioned, described and referenced in the present disclosure.

All of the documents cited herein are hereby incorporated in their entirety by reference and made an integral part of the present disclosure for all purposes.

We claim:

1. A method of treating target cells to deliver molecules to said cells, comprising
(a) exposing the cells to an aggregate composition which comprises VP22, or a polypeptide with the transport function of VP22, and oligonucleotides or polynucleotides; and
(b) exposing the cells to a disaggregating agent which can promote disaggregation of the aggregate composition in cells,
wherein steps (a) and (b) are carried out either simultaneously or sequentially, thereby delivering the molecules to the cells.

2. A method according to claim 1, wherein the VP22 protein, or the polypeptide with the transport function of VP22, is a fusion protein which also comprises a non-VP22 polypeptide sequence.

3. A method according to claim 1, wherein the VP22 protein, or the polypeptide with the transport function of VP22, is chemically cross linked to a non-VP22 molecule.

4. A method according to claim 1, wherein the oligonucleotide or polynucleotide comprises a circular plasmid.

5. A method according to claim 1, wherein the oligonucleotide or polynucleotide is linked to an additional molecule.

6. A method according to claim 1, wherein the disaggregating agent is a photoactivator and can promote disaggregation of the aggregate compositions following illumination with actinic light.

7. A method according to claim 6, wherein the disaggregating agent is a phthalocyanine-containing chromophore.

8. A method according to claim 1, wherein the disaggregating agent is an agent which can promote disaggregation in the absence of light.

9. A method according to claim 1, wherein the target cells are cells in vitro or in vivo.

10. A method according to claim 1, wherein the disaggregating agent is a photoactivator and wherein the method further comprises exposing the target cells to actinic light after delivery of the disaggregating agent.

11. A method according to claim 1, wherein the aggregates and disaggregating agent are administered separately to target cells in vivo at the same loci or at closely neighbouring loci.

12. A method according to claim 1, wherein the aggregates and disaggregating agent are administered as together as a combined preparation to target cells in vivo.

13. The method of claim 1, wherein delivering the molecules to the cells results in inhibiting the proliferation of the cells.

14. The method of claim 1, wherein delivering the molecules to the cells results in killing the cells.

15. A product comprising (a) an aggregate composition which comprises VP22, or a polypeptide with the transport function of VP22, and oligonucleotides or polynucleotides, and (b) a disaggregating agent which can promote disaggregation of the aggregate composition in cells, as a combined preparation for administration of the components (a) and (b) either sequentially or together, for use in treating cells by delivery of molecules to cells.

16. A method for treating a subject having a disease treatable by delivery of a molecule to a cell of the subject, comprising
administering to a cell of the subject the product of claim 15, thereby delivering the molecule to the cell and treating the subject.

17. A method of inhibiting cells from proliferating, comprising
administering to the cells the product of claim 15, thereby inhibiting the cells from proliferating.

18. The method of claim 17, wherein administering the product of claim 15 to the cells results in killing the cells.

19. A pharmaceutical comprising
(a) an aggregate composition which comprises VP22, or a polypeptide with the transport function of VP22, and oligonucleotides or polynucleotides; and
(b) a disaggregating agent which can promote disaggregation of the aggregate composition in cells,
in combination with a pharmaceutically acceptable excipient.

20. A cell preparation obtained by treating target cells in vitro according to the method of claim 1.

21. A cell preparation according to claim 20, in combination with a pharmaceutically acceptable excipient.
